(19) United States
(12) Patent Application Publication
(10) Pub. No.: US 2026/0298461 A1
(43) Pub. Date: Oct. 1, 2026

(54) LUMINAIRE PROVIDED WITH A UV LIGHTING MODULE

(71) Applicant: SIGNIFY HOLDING B.V., EINDHOVEN (NL)

(72) Inventors: TIES VAN BOMMEL, HORST (NL); REMY CYRILLE BROERSMA, HORST (NL)

(21) Appl. No.: 19/480,231

(22) PCT Filed: Apr. 16, 2024
(86) PCT No.: PCT/EP2024/060301

§ 371 Date: Oct. 30, 2025

(30) Foreign Application Priority Data
May 2, 2023 (EP) 23171149.0

(51) Int. Cl. F21V 33/00; F21S 8/04; F21V 5/00; F21V 21/28; F21Y 105/16; F21Y 113/13; F21Y 115/10; H05B 47/115; H05B 47/155

(52) U.S. Cl. CPC F21V 33/0064; F21S 8/04; F21V 5/007; F21V 21/28; H05B 47/115; H05B 47/155; F21Y 2105/16; F21Y 2113/13; F21Y 2115/10

(57) ABSTRACT

A luminaire system comprising a luminaire (1) including at least one luminaire white light source (3) associated with a respective luminaire collimator (8), wherein the collimated white light (32) has a main luminaire optical axis (OL) which, when the luminaire is mounted in or onto a ceiling (30), is aligned with a direction of gravity, and a connection slot. The system further comprises a UV lighting module (7) including a connection portion (11) configured to be inserted into the connection slot (6) so as to be releasably attachable, and a light emitting portion (12) housing a module solid state light source (13) configured to generate module light (34) comprising UV light having a (dominant) peak wavelength in the 280-320 nm range or in the 200-280 nm range and/or 320-380 nm range, and a light exit window (14) arranged to exit the module light (34) in a main module optical axis (OM), wherein the main module optical axis (OM) is different from the main luminaire optical axis (OL) and the light exit window (14) extends in a vertical plane when the luminaire is mounted in or onto a ceiling (30).

### FIELD OF THE INVENTION

The present invention relates to UV illumination, and particularly to a luminaire having a releasably attached UV lighting module.

### BACKGROUND OF THE INVENTION

UV light has properties beneficial for humans, such as promoting the production of vitamin B. UV light is a component of natural sunlight, but in modern society many people are confined to spending much of their time indoors. Artificial UV light can then be advantageous to maintain a healthy vitamin D production.

Artificial UV light may be provided in many ways and in many contexts. However, it would be beneficial to provide artificial UV light as an integrated part of general illumination, e.g. in an office space. In particular, it would be beneficial if existing luminaires could be easily retro-fitted to also provide UV lighting.

EP3660385A1 discloses a lighting device including a first light source emitting a visible light and a second light source spaced apart from the first light source and emitting light having a sterilization wavelength.

### SUMMARY OF THE INVENTION

It is an object of the present invention to provide a luminaire with improved UV lighting capabilities.

According to a first aspect of the invention, this and other objects are achieved by a luminaire system comprising a luminaire including at least one luminaire solid state light source configured to emit white light, each luminaire solid state light source being associated with a respective luminaire collimator arranged to collimate the white light into collimated white light, wherein the collimated white light has a main luminaire optical axis, OL, which, when the luminaire is mounted in or onto a ceiling, is aligned with a direction of gravity, and a connection slot. The system further comprises a UV lighting module including a connection portion configured to be inserted into the connection slot so as to be releasably attachable, and a light emitting portion housing a module solid state light source configured to generate module light comprising UV light having a (dominant) peak wavelength in the 280-320 nm range (UVB), and a light exit window arranged to exit the module light in a main module optical axis, OM, wherein the main module optical axis, OM, is different from the main luminaire optical axis, OL.

Alternatively, the module solid state light source may be configured to generate module light comprising UV light having a (dominant) peak wavelength in the 200-280 nm range (UVC) and/or 320-380 nm range (UVA).

White light is here intended broadly but may for example be light having a correlated colour temperature in the range 1500K-6500K, and a CRI of at least 80. The collimated white light may have a first full-width-half-max (FWHM1) and the UV light may have a second full-width-half-max (FWHM2), wherein FWHM1-FWHM2≥20 degrees.

With this system, a connection slot such as one intended for connection of sensor modules, is used to connect the UV lighting module to a white light luminaire.

In some embodiments, the spatial light distribution of the collimated white light may have a first full-width-half-max (FWHM1) equal to or less than 130 degrees, preferably FWHM1≤100 degrees, more preferably FWHM1≤80 degrees, most preferably FWHM1≤60 degrees. In this way, the collimated white light is office complaint and it is desired to reduce/avoid glare. Because UV light is invisible, glare is not an issue for the module light.

Connection slots for connecting various sensors to a luminaire are known e.g. the “upgradeable sensor slot” from Philips. The conventional understanding, and common practice, is that any module connected to such a slot has essentially the same dimensions as the slot. Such existing slots have therefore not been considered for UV-lighting, as a larger exit surface is desired when one wants to lower the (local) high intensity of UV light while maintaining the same amount of UV flux.

By separating the main optical axis of the UV light from the main optical axis of the collimated white light, the distribution of the UV light can be more flexible. For example, the UV light can be distributed transversely to the white light.

The main module optical axis, OM, can form an angle a of at least 20 degrees, preferably at least 45 degrees, more preferably at least 60 degrees, or more than 70 degrees and up to 90 degrees with respect to the main luminaire optical axis OL. An angle of 90 degrees means that, when the luminaire is mounted in a ceiling, the main module optical axis, OM, is substantially parallel to the ceiling.

The luminaire may have the feature that when the module is in inserted position in the connection slot of the luminaire and the luminaire is mounted in a ceiling, the module protrudes from the luminaire in the direction gravity slightly below the ceiling, such that, when in operation, the module provides over the ceiling, grazing module light, comprising UV light.

The light emitting portion may comprise a module collimator for collimating the module (UV) light along the module optical axis OM. For example, such a module collimator may be arranged in the light exit window.

When also the module light is collimated, it may have a broader or a more narrow spatial beam width than the collimated white light. For example, if a spatial light distribution of the collimated white light has a first full-width-half-max (FWHM1) and a spatial light distribution of the UV light has a second full-width-half-max (FWHM2), then the difference may be more than 20 degrees, i.e. |FWHM1-FWHM2|≥20 degrees.

In some embodiments, the spatial light distribution of the collimated module light may have a second full-width-half-max (FWHM2) equal to or less than 130 degrees, preferably FWHM2≤90 degrees, more preferably FWHM2≤50 degrees, most preferably FWHM2≤10 degrees.

In some embodiments, the spatial light distribution of the (collimated) module light may be higher than the spatial light distribution of the collimated white light.

In some embodiments, the collimated module light may have a bat-wing spatial light distribution.

The module collimator and luminaire collimator may differ in terms of (i) size, (ii) shape, (iii) reflectivity and/or (iv) material. In some embodiments, the module collimator is smaller than the luminaire collimator e.g. at least a factor two or three. The obtained effect is improved general light, because the module collimator does not (or less) block the (collimated) white light. In some embodiments, the shape of the module collimator more or less advanced (depending on if the UV light needs to be more or less collimated with respect to the collimated white light). In some embodiments, the reflectivity of the module collimator and luminaire collimator differ. Typically, the reflectivity in the UV(B) range is higher for the module collimator than for the luminaire collimator. In some embodiments, the material of the module collimator and luminaire collimator differ. Typically the material of the module collimator is made from a material with a high lifetime/durability for UV(B) light, which is not necessary for the luminaire collimator (and thus saving costs).

In some embodiments, the light emitting portion further comprises an optical element configured to diffuse or scatter the module light. This may be beneficial, especially if UV radiation is intended to illuminate a particular object, or if it is intended to be homogeneously spread in the ambient.

The light exit window may extend in a vertical plane when the luminaire is mounted in or onto a ceiling. Such a design will promote UV light emission transverse to the direction of gravity.

In some embodiments, at least a distal part of the light emitting portion (including the light exit window) is displaceable with respect to the connection portion between a first retracted position, in which the light exit window is at a first distance, D1, from the connection portion, and an extracted position, in which the light exit window is at a second distance, D2, from the connection portion, wherein the second distance, D2, is larger than the first distance, D1. The second distance D2 can be for example in the range 2-20 cm.

Separating the light exit window 14 from the connection portion may be advantageous, as the UV light emission is separated further from the ceiling, thereby reducing any negative impact of the UV(B) light on the ceiling surface (e.g. discoloring of paint, etc.).

In some embodiments, at least a distal part of the light emitting portion (including the light exit window) is rotatably arranged with respect to the connection portion about a rotation axis extending in the direction of gravity. The module optical axis, OM, can thereby be easily changed by rotating the distal part.

In some embodiments, at least a distal part of the light emitting portion is tiltably arranged with respect to the connection portion over a tilting angle, preferably 0 is in the range from 0 to 180 degrees, for example 0 to 90 degrees. 0 can be defined as the angle between the main luminaire optical axis OL and an elongation axis OD of the distal part. Again, this means that module optical axis, OM, can be easily changed by tilting the distal part.

In combination, rotation and tilting will provide full freedom to direct the UV light in any direction.

The UV lighting module may comprise a controller for individually controlling the luminaire light source and the module light source. Such a controller may further be functionally connected to an indicator (e.g. visual, light, etc.) and be configured to activate the indicator when the module light source is activated.

The module may further comprise a comprise a (proximity) sensor functionally connected to the controller and configured to provide a (proximity) sensor signal indicating presence of an object (such as a person or animal) in a field of view of the (proximity) sensor, wherein the controller is configured to control the module solid state light source in dependence on the (proximity) sensor signal. For example, the controller may be configured to dim, or even deactivate, the module solid state light source when there is no one in the field of view.

In some embodiments, the luminaire includes a central light emitting portion and a peripheral rim at least partly surrounding the central portion, and the connection slot is provided in this rim. This allows mounting a UV lighting module without obstructing the light from the luminaire. Preferably, the light exit window of the UV lighting device is elongated and has a longitudinal axis aligned with said rim. This ensures that the UV light emitting portion including the light exit window does not extend over the light emitting portion of the luminaire.

The connection slot is preferably configured to provide a mechanical connection for securing the UV lighting module in the luminaire, and an electrical connection for providing electrical power to the UV lighting module.

### BRIEF DESCRIPTION OF THE DRAWINGS

This and other aspects of the present invention will now be described in more detail, with reference to the appended drawings showing embodiments of the present invention.

FIG. 1 is a perspective view of a luminaire provided with a connection slot for receiving a UV lighting module according to an embodiment of the invention.

FIG. 2 shows the UV lighting module in FIG. 1 in more detail.

FIGS. 3A-D schematically shows different positioning of a UV lighting module according to further embodiments of the invention.

FIG. 4 shows a room provided with two luminaires as shown in FIG. 1.

### DETAILED DESCRIPTION

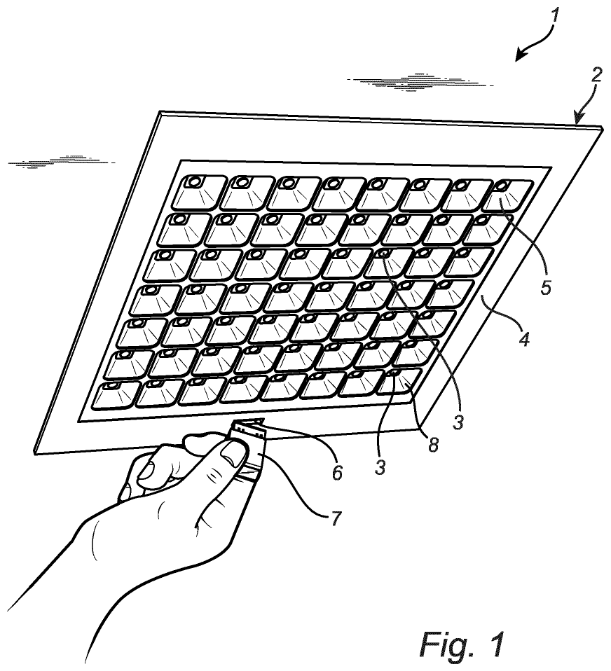

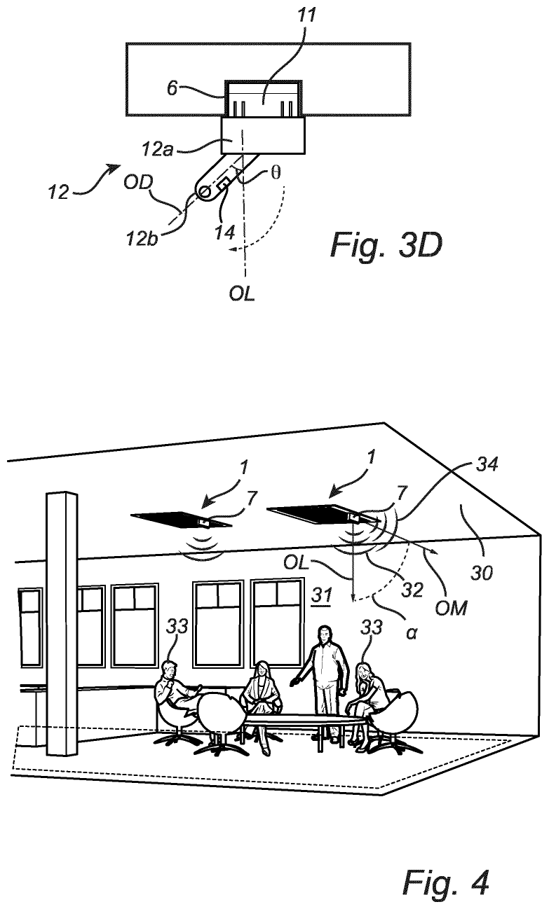

The luminaire 1 in FIG. 1 includes a housing 2 and a plurality of light sources 3 arranged in the housing 2. The light sources 3 are typically solid-state light sources such as LED light sources, configured to provide white light. The white light may have a correlated colour temperature in the range 1500K-6500K, and a CRI of at least 80. Each light source 3 is provided with a luminaire collimator 8, for providing office compliant collimated white light. When the luminaire 1 is mounted to the ceiling, the collimated white light is emitted in a direction along a main luminaire optical axis, OL (see FIG. 4), which substantially coincides with the direction of gravity.

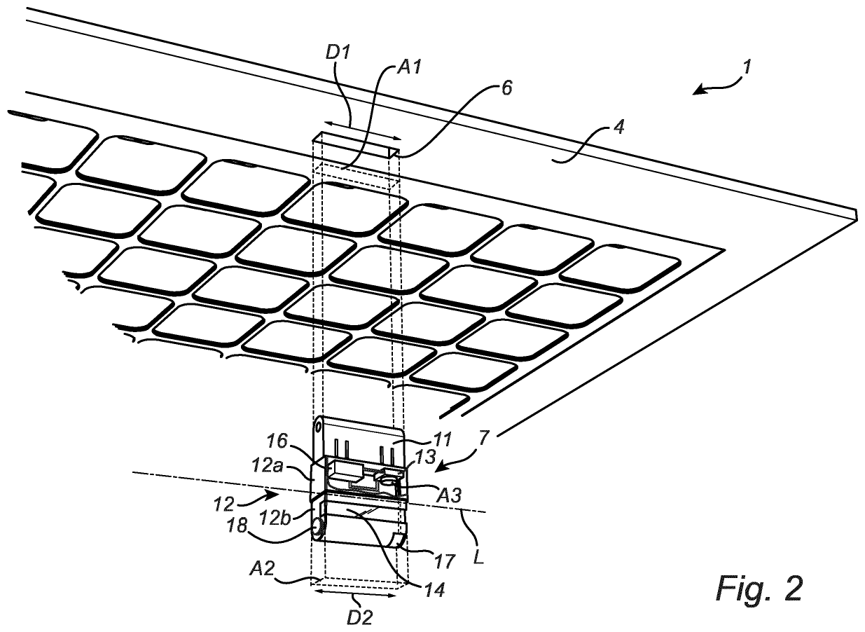

As shown in FIG. 1, the housing 2 forms a frame 4 surrounding a light generation portion 5 where the light sources 3 are arranged. The light sources 3 are typically configured to emit white, office-compliant light. In the frame 4 is provided a connection slot 6, configured to receive a plug-in module 7 such that the module 7 is releasably attachable to the luminaire 1. Various types of modules can be connected, including e.g. various sensor modules. Various luminaires with this type of “sensor slot” are sold by Philips ®. Each connection slot 6 has an opening to receive the plug-in module 7, and this opening has a width D1 (i.e. extension in the longest dimension) and an opening area A1 (see FIG. 2). As an example, the width may be a few centimeters, e.g. 1-8 cm, preferably 3-5 cm. The area A1 may be in the range 2-20 cm².

Releasable mechanical connection of module 7 in the sensor slot 6 may be provided in a snap-in manner, e.g. by means of a clip or bracket. Electrical connection of the module 7 may be provided by a galvanic plug-and-socket type connection, or by a wireless connection, e.g. through induction. Alternatively, the module 7 may be provided with an on-board power supply, such as a disposable or rechargeable battery (not shown). Details of suitable mechanical and electrical connections are well-known in the art, and will not be discussed in detail herein.

According to aspects of the present invention, the module 7 is a UV lighting module configured to emit UV light. This UV lighting module 7 will be described in more detail with reference to FIG. 2.

The UV lighting module 7 has a connection portion 11, configured to be inserted into the connection slot 6 so as to be mechanically secured therein. The UV lighting module 6 further has a UV light emitting portion 12, housing at least one solid state light source 13 configured to generate UV light (also referred to as module light), in this case having a dominant peak wavelength in the 280-320 nm range (UV). Alternatively, the solid state light source(s) 13 may be configured to light comprising UV light having a (dominant) peak wavelength in the 200-280 nm range (UVC) and/or 320-380 nm range (UVA).

The light emitting portion 12 has a light exit window 14, here formed in the vertical side of the light emitting portion 12. The exit window 14 is therefore configured to exit the UV light into the ambient in a direction along a main module optical axis, OM, different from the direction OL (see FIG. 4). The light emitting portion 12 may operate as a light mixing chamber, in which case the exit window 14 is a transparent cover, e.g. plastic or glass. Alternatively, the exit window 14 is simply an opening.

In some embodiments, the light emitting portion 12 further houses at least one (further) module solid state light source (not shown) configured to generate further module light comprising violet light having a dominant peak wavelength in the 380-420 nm range. Alternatively, or in addition, in an embodiment the at least one (further) module solid state light source may provide colored light.

The light exit window 14 may be adapted to, or may comprise a separate optical element configured to, diffuse or scatter the light from the LEDs 13. The light exit window 14 may also be curved, to provide an improved distribution of emitted light.

Specifically, the exit window 14 may serve as a module collimator, in which case also the UV light is collimated. The collimation of the UV light may be stronger or weaker than the white light collimation. In other words, the beam-width of the UV light may be smaller or greater than the beam-width of the white light.

The window area A2 of the light exit window 14 is here illustrated as comparable in size as the slot opening area A1, but may in some embodiments be larger, e.g. at least 1.5 times larger. Each LED 13 has a light output surface area A3 which may be less than ⅕ of the opening area A1.

A controller 16 is connected to control the LEDs 13, 15.

In the illustrated example, the LED module 6 further comprises a proximity sensor 17, e.g. an IR sensor or UWB radar sensor. The sensor can be configured to detect when an object is in a field of view of the sensor 17. Alternatively, the sensor can be configured to detect movement within the field of view. The sensor 17 may also be connected to the controller 16, to thereby provide proximity-based control of the emitted light. For example, the controller may be configured to only activate the LEDs 13, 15 in a situation where a living being is determined to be in a field of view of the sensor 17. This may avoid UV emission when there is no-one in a room.

The module 7 may also comprise an indicator 18, e.g. a visible light indicator, connected to the controller 16. The controller can be configured to activate the indicator 18 whenever the UV light source 13 is activated, thereby making a person in the vicinity aware of the UV light emission.

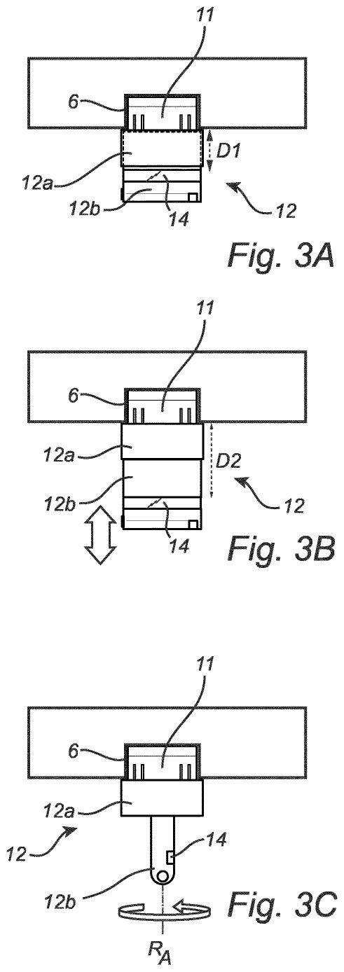

In some embodiments, the UV lighting module 7 is configured to be oriented in different ways, in order to modify the main module optical axis, OM. This is illustrated schematically in FIGS. 3A-D. In these figures, the light emitting portion 12 is illustrated as being formed by two parts 12a, 12b, which are telescopically arranged in each other. A base part 12a connected to the connecting portion 11, and a distal part 12b, in which the exit window 14 is provided. The distal part 12b may also be rotatable and/or pivotable with respect to the base part 12a.

FIG. 3A illustrates the UV lighting module 7 connected to a connection slot 6, and where the distal part 12b is fully inserted into the base part 12a in a retracted position. In this configuration, the light exit window 14 is at a first distance D1 from the connection portion 11 (and form the ceiling).

In FIG. 3B, the distal part 12a (with the light exit window 14) has been extracted—downwards in the drawing—so that the exit window is at a distance D2 from the connection portion 11 (and from the ceiling). The ceiling next to the UV lighting module 7 is thereby subject to less intense UV(B) light.

In FIG. 3C, the distal part 12b has been rotated with respect to the base part 12a, around a vertical axis RA, so that the UV light is emitted in a different direction in the same plane. In the illustrated case, the distal part 12 b has been rotated 90 degrees.

Finally, in FIG. 3D, the distal part 12b has a main distal part axis OD and has been pivoted with respect to the base part 12a, so that UV light can be emitted in a direction forming a different angle 0 of the main distal part axis OD with respect to the direction of gravity (vertical) (here along the main luminaire optical axis OL).

FIG. 4 shows two luminaires 1 provided with UV lighting modules 7 suspended in a ceiling 30 of a room 31. Collimated white light 32 is emitted from the luminaires 1 in a direction OL downwards into the room 31. At the same time, UV(B) light 34 is emitted from the UV lighting modules 7 in a direction OM sideways and at an angle a with respect to the white light 32. The UV(B) light 34 is reflected around the room 31 and ensures a healthy vitamin D production in people present in the room.

The person skilled in the art realizes that the present invention by no means is limited to the preferred embodiments described above. On the contrary, many modifications and variations are possible within the scope of the appended claims. For example, other types of luminaires, provided with appropriate connection slots, can be combined with a UV lighting module according to the invention. Also, other solutions for redirection of the module optical axis OM are possible, other than those shown in FIGS. 3A-D.

1. A luminaire system comprising:
(i) a luminaire including at least one luminaire solid state light source (configured to emit white light, each luminaire solid state light source being associated with a respective luminaire collimator arranged to collimate said white light into collimated white light, wherein the collimated white light has a main luminaire optical axis which, when said luminaire is mounted in or onto a ceiling, is in the direction of gravity, and a connection slot; and
(ii) a UV lighting module including a connection portion configured to be inserted into said connection slot so as to be releasably attachable to said connection slot, and a light emitting portion housing a module solid state light source configured to generate module light comprising UV light having a dominant peak wavelength in the 280-320 nm range or in the 200-280 nm range and/or 320-380 nm range, and a light window arranged to exit the module light along a main module optical axis;
wherein said main module optical axis is different from said main luminaire optical axis, and
wherein the light exit window extends in a vertical plane when the luminaire is mounted in or onto a ceiling.

2. The luminaire system according to claim 1, wherein said main module optical axis forms an angle a of 90 degrees with respect to said main luminaire optical axis.

3. The luminaire system according to claim 2, wherein, when said luminaire is mounted in or onto a ceiling, said main module optical axis is substantially parallel to the ceiling.

4. The luminaire system according to claim 1, wherein the light emitting portion further comprises a module collimator configured to collimate the module light as collimated module light along said main module optical axis optionally the light exit window serves as a module collimator.

5. The luminaire system according to claim 4, wherein a spatial light distribution of the collimated white light has a first full-width-half-max (FWHM1) and a spatial light distribution of the collimated module light has a second full-width-half-max (FWHM2), wherein |FWHM1-FWHM2|≥20 degrees.

6. The luminaire system according to claim 4, wherein module collimator and luminaire collimator differ in terms of at least one of (i) size, (ii) shape, (iii) reflectivity and (iv) material.

7. The luminaire system according to claim 1, wherein an exit surface of the light exit window is larger than the dimensions of the slot.

8. The luminaire system according to claim 1, wherein at least a distal part of said light emitting portion is displaceable with respect to said connection portion between a first retracted position, in which said light exit window is at a first distance from the connection portion, and an extracted position, in which said light exit window is at a second distance from the connection portion, wherein the second distance is larger than the first distance.

9. The luminaire system according to claim 1, wherein at least a distal part of said light emitting portion is rotatably arranged with respect to said connection portion about a rotation axis extending in the direction of gravity.

10. (canceled)

11. The luminaire system according to claim 1, further comprising a controller for individually controlling white light emission from the luminaire light source and module light emission from the module solid state light source.

12. The luminaire system according to claim 11, further comprising a proximity sensor functionally connected to the controller and configured to provide a proximity sensor signal indicating presence of an object in a field of view of the proximity sensor, wherein the controller is configured to control the module solid state light source in dependence on the proximity sensor signal.

13. The luminaire system according to claim 1, wherein the luminaire includes a central light generation portion and a peripheral rim at least partly surrounding the central light generation portion, and wherein the connection slot is provided in said rim.

14. The luminaire system according to claim 1, wherein the connection slot is configured to provide a mechanical connection for releasably securing said UV lighting module in the luminaire and/or wherein the connection slot is configured to provide electrical power to the UV lighting module.

15. The luminaire system according to claim 1, wherein said UV light emitting portion further comprises a further module solid state light source configured to generate further module light comprising violet light having a dominant peak wavelength in the 380-420 nm range.
